(19) United States
(12) Patent Application Publication
(10) Pub. No.: US 2025/0177634 A1
(43) Pub. Date: Jun. 5, 2025

(54) CAP FOR CONTROLLING FLUID FLOW

(71) Applicant: Mizuho Miortal MORRISON, Newport Beach, CA (US)

(72) Inventor: Mizuho Miortal MORRISON, Newport Beach, CA (US)

(73) Assignee: STAT CAP LLC, Las Vegas, NV (US)

(21) Appl. No.: 18/837,497

(22) PCT Filed: Feb. 9, 2023
(86) PCT No.: PCT/US2023/062300

§ 371 Date: Aug. 9, 2024

Related U.S. Application Data
(60) Provisional application No. 63/309,281, filed on Feb. 11, 2022.

(51) Int. Cl. A61M 3/02; B65D 47/20

(52) U.S. Cl. CPC A61M 3/0279; A61M 3/0245; B65D 47/20; A61M 2205/3334

(57) ABSTRACT

The present disclosure relates to a flow control device, including a body with a connecting portion. The body includes a structure defining at least one or more pre-existing fluid channels. The at least one or more pre-existing fluid channels can be configured to control a fluid flow rate of a fluid exiting the body. Controlling the fluid flow rate can include controlling a predetermined pressure of the fluid flow rate. The connecting portion can attach to a container.

### CROSS-REFERENCE TO RELATED APPLICATIONS

This application claims the benefit of and priority to U.S. Provisional Patent Application No. 63/309,281, filed Feb. 11, 2022, and entitled “Cap for Controlling Fluid Flow,” which is hereby incorporated by reference in its entirety.

### FIELD

The present disclosure relates to containers with a cap in a medical environment and uses thereof.

### BACKGROUND

Generally, a container can have a cap. The cap for the container can be used to prevent the contents from exiting the container. Generally, to remove the contents of the container, the cap may need to be removed from the container (e.g., unscrew the cap from the container, etc.). Generally, once the cap is removed, the contents can be removed from the container.

### SUMMARY

Irrigation of open wounds can be a standard practice in many medical environments. From emergency departments and operating rooms to outpatient primary care and wound care clinics, some wounds require irrigation as part of the wound management process. For traumatic open wounds (e.g., lacerations, animal bites, puncture wounds, etc.), irrigation with sterile saline or water can be needed to remove foreign material (e.g., bacteria, soil, other particulate matter, etc.) before wound closure. Removal of the foreign material can help reduce infection rates of the wound. Irrigation is also warranted in medical, sterile environments where sterile surgical incisions may require irrigation before wound closure to help ensure proper healing of the wound. In some embodiments, medical, sterile environments can include emergency department rooms, operating rooms, outpatient primary care clinics, wound care clinics, etc. The irrigation before wound closure may be needed for several medical reasons, including removal of blood and debris, etc.

The present disclosure relates to a cap with pre-existing fluid channels to control fluid flow for irrigating wounds. According to some embodiments, the cap has a universal fit for attaching to a container (e.g., a saline/water bottle, etc.). With the cap attached to the container, a device can be formed to create a spray bottle (e.g., a sterile spray bottle, etc.). The configuration of the pre-existing fluid channels, including the number of pre-existing fluid channels and the arrangement of the pre-existing fluid channels on the cap, provides for a controlled fluid flow rate and a defined surface area to increase the effectiveness of irrigating the wounds. In some embodiments, the present disclosure can reduce wound preparation time by quickly, safely, and efficiently irrigating a wound.

In some embodiments, the techniques described herein relate to a flow control device including: a body with a connecting portion, wherein the body includes: a structure defining at least one or more pre-existing fluid channels, wherein the at least one or more pre-existing fluid channels are configured to control a fluid flow rate of a fluid exiting the body, and wherein controlling the fluid flow rate includes controlling a predetermined pressure of the fluid flow rate; and wherein the connecting portion is configured to attach to a container.

In some embodiments, the techniques described herein relate to a flow control device, wherein the predetermined pressure ranges from about 5 pounds/square inch (psi) to about 15 psi.

In some embodiments, the techniques described herein relate to a flow control device, wherein the body includes a first surface and a second surface.

In some embodiments, the techniques described herein relate to a flow control device, wherein the at least one or more pre-existing fluid channels include a first plurality of pre-existing fluid channels on the first surface and a second plurality of pre-existing fluid channels on the second surface.

In some embodiments, the techniques described herein relate to a flow control device, wherein a cross-sectional area of each fluid channel of the first plurality of pre-existing fluid channels is the same.

In some embodiments, the techniques described herein relate to a flow control device, wherein a cross-sectional area of each fluid channel of the first plurality of pre-existing fluid channels is different than a cross-sectional area of each fluid channel of the second plurality of pre-existing fluid channels.

In some embodiments, the techniques described herein relate to a flow control device, wherein the second surface is at least partially recessed on the body defining an interior well on the body.

In some embodiments, the techniques described herein relate to a flow control device, wherein the second surface of the body is substantially perpendicular to a longitudinal axis of the body.

In some embodiments, the techniques described herein relate to a flow control device, wherein the flow control device is configured to be used in a medical, sterile environment.

In some embodiments, the techniques described herein relate to a flow control device, wherein the connecting portion is threaded.

In some embodiments, the techniques described herein relate to a container including any flow control device as described herein.

In some embodiments, the techniques described herein relate to a container, wherein the container is configured to contain the fluid prior to the fluid exiting the body.

In some embodiments, the techniques described herein relate to a container including: a main body; a flow control device connected to the main body, wherein the flow control device includes: a body of the flow control device with a connecting portion, wherein the body of the flow control device includes: a structure defining at least one or more pre-existing fluid channels, wherein the at least one or more pre-existing fluid channels are configured to control a fluid flow rate of a fluid exiting the body of the flow control device, and wherein controlling the fluid flow rate includes controlling a predetermined pressure of the fluid flow rate; and wherein the connecting portion is configured to attach to the container.

In some embodiments, the techniques described herein relate to a container, wherein the container is configured to contain the fluid.

In some embodiments, the techniques described herein relate to a method including: connecting a fluid control device to a container, wherein the fluid control device includes: a body with a connecting portion, wherein the body includes: a structure defining at least one or more pre-existing fluid channels, wherein the at least one or more pre-existing fluid channels are configured to control a fluid flow rate of a fluid exiting the body, and wherein controlling the fluid flow rate includes controlling a predetermined pressure of the fluid flow rate; and wherein the connecting portion is configured to attach to the container; and delivering the fluid to a site from the container at a controlled fluid flow rate.

In some embodiments, the techniques described herein relate to a method, wherein delivering the fluid to the site from the container at the controlled fluid flow rate includes deforming the container.

In some embodiments, the techniques described herein relate to a method, wherein deforming the container includes compressing the container.

In some embodiments, the techniques described herein relate to a method, wherein delivering the fluid to the site from the container at the controlled fluid flow rate includes delivering the fluid at the predetermined pressure.

In some embodiments, the techniques described herein relate to a method, wherein the predetermined pressure ranges from about 5 psi to about 15 psi.

In some embodiments, the techniques described herein relate to a method including: connecting a fluid control device to a container holding a sterile fluid, wherein the fluid control device includes: a body with a connecting portion, wherein the body includes: a structure defining at least one or more pre-existing fluid channels, wherein the connecting portion is configured to attach to the container; delivering the sterile fluid to a site from the container at a controlled fluid flow rate while maintaining sterility of the sterile fluid.

In some embodiments, the techniques described herein relate to a method, wherein delivering the sterile fluid to the site comprises delivering the sterile fluid to an irrigation coverage area of the site ranging from 5,000 mm² to about 45,000 mm².

In some embodiments, the techniques described herein relate to a kit including: a flow control device including: a body with a connecting portion, wherein the body includes: a structure defining at least one or more pre-existing fluid channels, wherein the at least one or more pre-existing fluid channels are configured to control a fluid flow rate of a fluid exiting the body, and wherein controlling the fluid flow rate includes controlling a predetermined pressure of the fluid flow rate; and wherein the connecting portion is configured to attach to a container; and a sealed package providing a sterile environment, wherein the flow control device is in the sterile environment of the sealed package.

In some embodiments, the techniques described herein relate to a kit, wherein the body includes a first surface and a second surface.

In some embodiments, the techniques described herein relate to a kit, wherein the at least one or more pre-existing fluid channels include a first plurality of pre-existing fluid channels on the first surface and a second plurality of pre-existing fluid channels on the second surface.

### BRIEF DESCRIPTION OF THE DRAWINGS

FIG. 1A is a top view of the flow control device of the present disclosure, in accordance with some embodiments.

FIG. 1B is a cross-sectional view of the flow control device of the present disclosure in FIG. 1A, in accordance with some embodiments.

FIG. 1C is a side view of the flow control device of the present disclosure in FIG. 1A, in accordance with some embodiments.

FIG. 2 displays a flow control device connected to a container, in accordance with some embodiments.

FIG. 3 depicts a kit including a flow control device in a sealed environment, in accordance with some embodiments.

FIG. 4A is a top view of a flow control device, in accordance with some embodiments.

FIG. 4B is a side view of the flow control device of the present disclosure in FIG. 4A, in accordance with some embodiments.

FIG. 4C is a bottom view of the flow control device of the present disclosure in FIG. 4A, in accordance with some embodiments.

FIG. 5 is a top perspective view of a flow control device attached to a container, in accordance with some embodiments.

FIG. 6 depicts a kit including a flow control device in a sealed environment, in accordance with some embodiments.

### DETAILED DESCRIPTION

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

Throughout the specification, the meaning of “a,” “an,” and “the” include plural references. The meaning of “in” includes “in” and “on.”

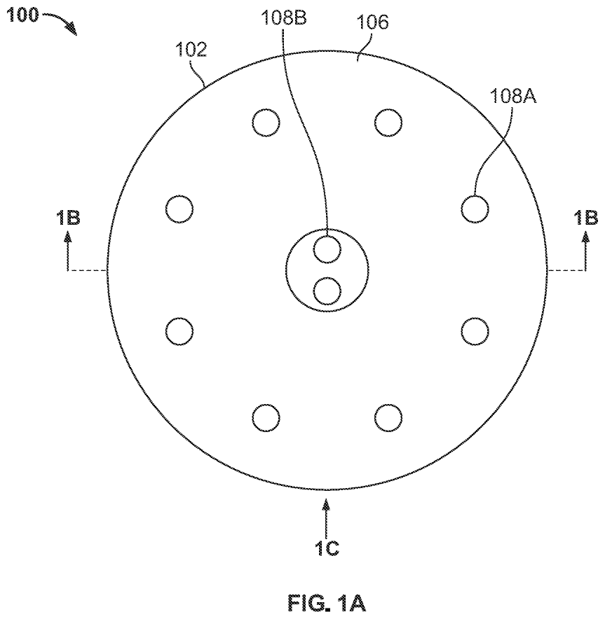

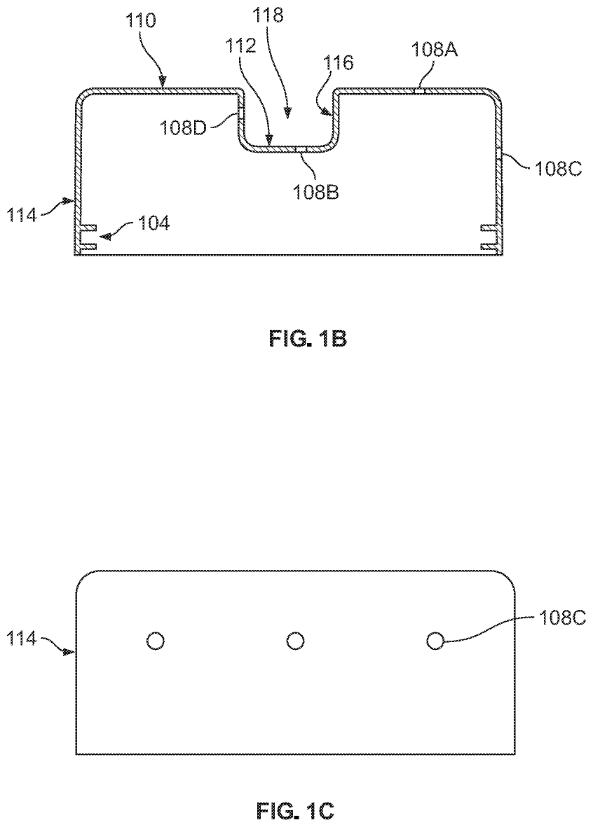

FIG. 1A is a top view of a flow control device 100 of the present disclosure, in accordance with some embodiments. FIG. 1B is a cross-sectional view of the flow control device 100 of the present disclosure in FIG. 1A, in accordance with some embodiments. FIG. 1C is an external, side view of the flow control device 100 of the present disclosure in FIG. 1A, in accordance with some embodiments.

The flow control device 100 includes a body 102 with a connecting portion 104. The connecting portion 104 is configured to attach to another apparatus (e.g., a container or a tube, etc.). The body 102 includes a structure 106.

The exterior of the body 102 includes a first surface 110, a second surface 112, a third surface 114, and a fourth surface 116. The second surface 112 is at least partially recessed on the body 102 defining an interior well 118 on the body 102. The fourth surface 116 defines a sidewall of the well 118. In some embodiments, the first surface 110 and the second surface 112 are substantially parallel to one another. In some embodiments, the third surface 114 and the fourth surface 116 are substantially parallel to one another. In some embodiments, the second surface 112 is substantially perpendicular to a longitudinal axis of the body 102.

In some embodiments, the structure 106 defines a first plurality of pre-existing fluid channels 108A on the first surface 110, a second plurality of pre-existing fluid channels 108B on the second surface 112, a third plurality of pre-existing fluid channels 108C on the third surface 114, and a fourth plurality of pre-existing fluid channels 108D on the fourth surface 116 (collectively referred to as a plurality of pre-existing fluid channels 108). FIG. 1C displays the third surface 114 with the third plurality of pre-existing fluid channels 108C.

The structure 106 defines at least one or more pre-existing fluid channels 108. The at least one or more pre-existing fluid channels 108 are configured to control a fluid flow rate of a fluid exiting the body 102. In some embodiments, controlling the fluid flow rate includes controlling a predetermined pressure of the fluid flow rate. In some embodiments, the predetermined pressure ranges from about 1 psi to about 20 psi, or any intervening number or range. For example, in some embodiments, the predetermined pressure ranges from about 5 psi to about 15 psi. In some embodiments, the predetermined pressure is more than about 5 psi. In some embodiments, the predetermined pressure is more than about 10 psi. In some embodiments, the predetermined pressure is more than about 15 psi. In some embodiments, the predetermined pressure is less than about 20 psi. In some embodiments, the predetermined pressure is less than about 15 psi. In some embodiments, the predetermined pressure is less than about 10 psi. In some embodiments, the predetermined pressure is less than about 5 psi. In some embodiments, the predetermined pressure ranges from about 5 psi to about 20 psi. In some embodiments, the predetermined pressure ranges from about 5 psi to about 10 psi. In some embodiments, the predetermined pressure ranges from about 1 psi to about 15 psi. In some embodiments, the predetermined pressure ranges from about 1 psi to about 10 psi.

The predetermined pressure selected is determined by being able to sufficiently irrigate the wound without causing harm to the tissue of the patient. In some embodiments, the predetermined pressure is measured as a fluid is exiting the at least one or more pre-existing fluid channels 108. In some embodiments, the predetermined pressure is measured at the site of where the fluid is directed (e.g., the wound site, etc.).

The at least one or more pre-existing fluid channels 108 can be a variety of shapes and sizes. In some embodiments, the shape of the at least one or more pre-existing fluid channels 108 can be symmetrical, non-symmetrical, geometric, or non-geometric. Geometric shapes include oval, circular, square, rectangular, or triangular. In some embodiments, the size of the at least one or more pre-existing fluid channels 108 ranges from about 23 gauge (0.642 mm) to about 14 gauge (2.109 mm), or any intervening range or value. In some embodiments, the size of the at least one or more pre-existing fluid channels 108 ranges from about 23 gauge (0.642 mm) to about 14 gauge (2.109 mm). In some embodiments, the size of the at least one or more pre-existing fluid channels 108 ranges from about 22 gauge (0.718 mm) to about 14 gauge (2.109 mm). In some embodiments, the size of the at least one or more pre-existing fluid channels 108 ranges from about 21 gauge (0.819 mm) to about 14 gauge (2.109 mm). In some embodiments, the size of the at least one or more pre-existing fluid channels 108 ranges from about 20 gauge (0.908 mm) to about 14 gauge (2.109 mm). In some embodiments, the size of the at least one or more pre-existing fluid channels 108 ranges from about 19 gauge (1.067 mm) to about 14 gauge (2.109 mm). In some embodiments, the size of the at least one or more pre-existing fluid channels 108 ranges from about 18 gauge (1.27 mm) to about 14 gauge (2.109 mm). In some embodiments, the size of the at least one or more pre-existing fluid channels 108 ranges from about 17 gauge (1.473 mm) to about 14 gauge (2.109 mm). In some embodiments, the size of the at least one or more pre-existing fluid channels 108 ranges from about 16 gauge (1.651 mm) to about 14 gauge (2.109 mm). In some embodiments, the size of the at least one or more pre-existing fluid channels 108 ranges from about 15 gauge (1.829 mm) to about 14 gauge (2.109 mm). In some embodiments, the size of the at least one or more pre-existing fluid channels 108 ranges from about 23 gauge (0.642 mm) to about 15 gauge (1.829 mm). In some embodiments, the size of the at least one or more pre-existing fluid channels 108 ranges from about 23 gauge (0.642 mm) to about 16 gauge (1.651 mm). In some embodiments, the size of the at least one or more pre-existing fluid channels 108 ranges from about 23 gauge (0.642 mm) to about 17 gauge (1.473 mm). In some embodiments, the size of the at least one or more pre-existing fluid channels 108 ranges from about 23 gauge (0.642 mm) to about 18 gauge (1.27 mm). In some embodiments, the size of the at least one or more pre-existing fluid channels 108 ranges from about 23 gauge (0.642 mm) to about 19 gauge (1.067 mm). In some embodiments, the size of the at least one or more pre-existing fluid channels 108 ranges from about 23 gauge (0.642 mm) to about 20 gauge (0.908 mm). In some embodiments, the size of the at least one or more pre-existing fluid channels 108 ranges from about 23 gauge (0.642 mm) to about 21 gauge (0.819 mm). In some embodiments, the size of the at least one or more pre-existing fluid channels 108 ranges from about 23 gauge (0.642 mm) to about 22 gauge (0.718 mm). In some embodiments, the size of the at least one or more pre-existing fluid channels 108 is about 18 gauge (1.27 mm).

In some embodiments, a cross-sectional area of each fluid channel of the at least one or more pre-existing fluid channels 108 are the same. In some embodiments, a cross-sectional area of each fluid channel of the at least one or more pre-existing fluid channels 108 are different. In some embodiments, a cross-sectional area of at least some of the fluid channels of the at least one or more pre-existing fluid channels 108 are the same. For example, a cross-sectional area of each fluid channel of the first plurality of pre-existing fluid channels 108A can be different than a cross-sectional area of each fluid channel of the second plurality of pre-existing fluid channels 108B.

In some embodiments, each fluid channel of the first plurality of pre-existing fluid channels 108A can be the same or different from one another. For example, if there are four fluid channels of the first plurality of pre-existing fluid channels 108A, two fluid channels can be the same or substantially similar and two can be different from the other fluid channels. Similarly, each fluid channel of the second plurality of pre-existing fluid channels 108B can be the same or different from one another. Each fluid channel of the third plurality of pre-existing fluid channels 108C can be the same or different from one another. Each fluid channel of the fourth plurality of pre-existing fluid channels 108D can be the same or different from one another.

In some embodiments, either one or more of the first plurality of pre-existing fluid channels 108A, the second plurality of pre-existing fluid channels 108B, the third plurality of pre-existing fluid channels 108C, or the fourth plurality of pre-existing fluid channels 108D are not present. For example, in some embodiments, the fourth plurality of pre-existing fluid channels 108D are not present. In some embodiments, the third plurality of pre-existing fluid channels 108C and the fourth plurality of pre-existing fluid channels 108D are not present.

In some embodiments, the number of fluid channels for the first plurality of pre-existing fluid channels 108A, the second plurality of pre-existing fluid channels 108B, the third plurality of pre-existing fluid channels 108C, or the fourth plurality of pre-existing fluid channels 108D can be the same or differ from one another. For each of the plurality of pre-existing fluid channels (108A, 108B, 108C, and 108D), there can be at least one fluid channel, thirty or less fluid channels, a range from two to thirty channels, or any intervening number. For example, there can be seven fluid channels of the first plurality of pre-existing fluid channels 108A, ten fluid channels of the second plurality of pre-existing fluid channels 108B, fourteen fluid channels of the third plurality of pre-existing fluid channels 108C, and thirty fluid channels of the fourth plurality of pre-existing fluid channels 108D. In another example, there can be ten fluid channels of the first plurality of pre-existing fluid channels 108A, ten fluid channels of the second plurality of pre-existing fluid channels 108B, zero fluid channels for the third plurality of pre-existing fluid channels 108C, and zero fluid channels of the fourth plurality of pre-existing fluid channels 108D. These examples are not exhaustive and are exemplary only. There can any numerical combination possible for the at least one or more pre-existing fluid channels 108.

In some embodiments, the flow control device 100 is configured to be used in a medical, sterile environment. The flow control device 100 can also be used in other applications, besides a medical, sterile environment. For example, the flow control device 100 can be attached to a fluid container for a user to drink from. The flow control device 100 can also be used in an industrial or residential environment. For example, the flow control device 100 can be connected to a tube to be used as a shower head.

The connecting portion 104 can connect the flow control device 100 to another apparatus, such as a device like a container, via a variety of connecting methods. For example, the connecting portion 104 can be a threaded portion, a friction-fit, snap-fit, adhesive, or lock and key, among others.

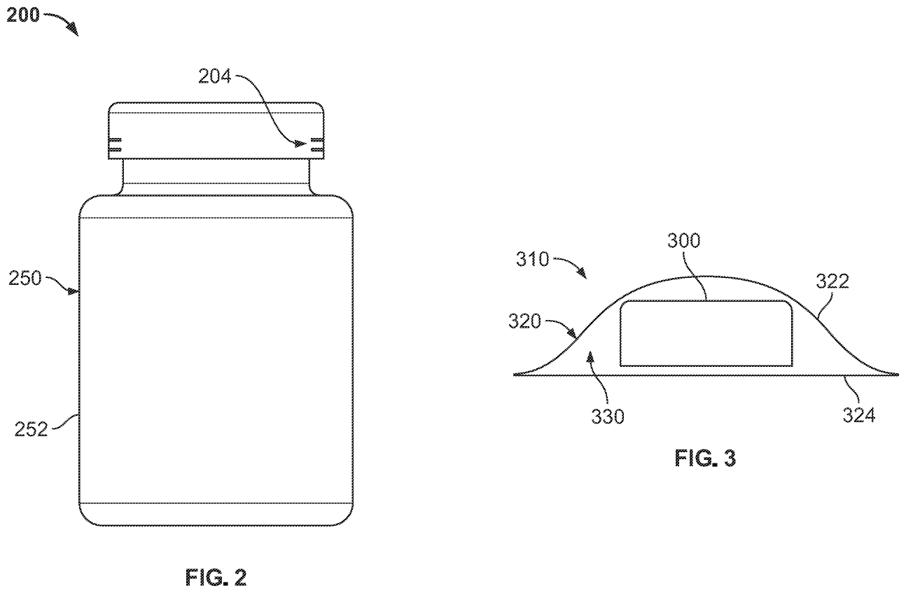

FIG. 2 displays a flow control device 200 connected to a container 250, in

accordance with some embodiments. The flow control device 200 is the same or similar to the flow control device 100. Accordingly, similar reference numbers are used. Some of the differences between the flow control devices 100 and 200 are discussed. However, it will be appreciated that other differences may exist without departing from the scope of this disclosure. As discussed herein, the container 250 can be any apparatus capable of holding a fluid. In some embodiments, the container 250 is defined by a main body 252 of the container 250. The flow control device 200 connects to the main body 252. A connecting portion 204 on an interior of the flow control device 200 is configured to attach to the container 250.

The container 250 is configured to contain the fluid. In some embodiments, the container 250 has a volume of 250 ml, 500 ml, or 1000 ml.

In some embodiments, a method of using the present disclosure (all references will be made to the flow control device 100 but any flow control device as described herein can be used) includes connecting the fluid control device 100 to the container 250 and delivering the fluid to a site (e.g., a wound site, etc.) from the container 250 at a controlled fluid flow rate.

In some embodiments, delivering the fluid to the site from the container 250 at the controlled fluid flow rate includes deforming the container 250. In some embodiments, deforming the container 250 includes compressing the container 250.

In some embodiments, delivering the fluid to the site from the container 250 at the controlled fluid flow rate includes delivering the fluid at the predetermined pressure. The predetermined pressure of the present disclosure, as discussed herein and above, can vary. The predetermined pressure ranges discussed herein, above, will not be repeated here. As an example, in some embodiments, the predetermined pressure ranges from about 5 psi to about 15 psi.

In some embodiments, a method of using the present disclosure (all references will be made to the flow control device 100 but any flow control device as described herein can be used) includes connecting the fluid control device 100 to a container 250 holding a sterile fluid and delivering the sterile fluid to a site from the container 250 at a controlled fluid flow rate while maintaining sterility of the sterile fluid. Discussion related to the flow control device 100 and the method of use apply to whether a sterile or non-sterile fluid is used.

In some embodiments, delivering the sterile fluid to the site includes delivering the sterile fluid to an irrigation coverage area of the site ranging from about 2,500 mm² to about 45,000 mm², or any intervening range or number. For example, in some embodiments, irrigation coverage area of the site ranges from about 10,000 mm² to about 45,000 mm². In some embodiments, irrigation coverage area of the site ranges from about 15,000 mm² to about 45,000 mm². In some embodiments, irrigation coverage area of the site ranges from about 20,000 mm² to about 45,000 mm². In some embodiments, irrigation coverage area of the site ranges from about 25,000 mm² to about 45,000 mm². In some embodiments, irrigation coverage area of the site ranges from about 30,000 mm² to about 45,000 mm². In some embodiments, irrigation coverage area of the site ranges from about 35,000 mm² to about 45,000 mm². In some embodiments, irrigation coverage area of the site ranges from about 40,000 mm² to about 45,000 mm². In some embodiments, irrigation coverage area of the site ranges from about 2,500 mm² to about 40,000 mm². In some embodiments, irrigation coverage area of the site ranges from about 2,500 mm² to about 35,000 mm². In some embodiments, irrigation coverage area of the site ranges from about 2,500 mm² to about 30,000 mm². In some embodiments, irrigation coverage area of the site ranges from about 2,500 mm² to about 25,000 mm². In some embodiments, irrigation coverage area of the site ranges from about 2,500 mm² to about 20,000 mm². In some embodiments, irrigation coverage area of the site ranges from about 2,500 mm² to about 15,000 mm². In some embodiments, irrigation coverage area of the site ranges from about 2,500 mm² to about 10,000 mm². In some embodiments, irrigation coverage area of the site ranges from about 2,500 mm² to about 5,000 mm². In some embodiments, irrigation coverage area of the site ranges from about 5,000 mm² to about 35,000 mm². In some embodiments, irrigation coverage area of the site ranges from about 10,000 mm² to about 35,000 mm². In some embodiments, irrigation coverage area of the site ranges from about 15,000 mm² to about 35,000 mm². In some embodiments, irrigation coverage area of the site ranges from about 15,000 mm² to about 30,000 mm². In some embodiments, irrigation coverage area of the site ranges from about 20,000 mm² to about 35,000 mm². In some embodiments, irrigation coverage area of the site ranges from about 20,000 mm² to about 30,000 mm².

Directing fluid at the irrigation coverage area of the site can be achieved while causing little to no further injury or harm to the patient. The user can hold the flow control device 100 at a distance of approximately from 25 mm (approximately one inch) to 900 mm (approximately 3 feet) away, or any intervening distance or distance range, from the wound site when directing fluid at the irrigation coverage area. For example, the user can hold the flow control device 100 away from the wound at a distance of about 25 mm to 800 mm, 25 mm to 700 mm, 25 mm to 600 mm, 25 mm to 500 mm, 25 mm to 400 mm, 25 mm to 300 mm, 25 mm to 200 mm, 25 mm to 100 mm, 25 mm to 50 mm, 50 mm to 900 mm, 100 mm to 900 mm, 200 mm to 900 mm, 300 mm to 900 mm, 400 mm to 900 mm, 500 mm to 900 mm, 600 mm to 900 mm, 700 mm to 900 mm, 800 mm to 900 mm, at least 25 mm, or less than 900 mm.

A large irrigation coverage area as described herein can be achieved without excessive pressures (see discussion of predetermined pressures described herein) that may cause further injury or harm to the patient. For example, in some embodiments, the flow control device 100 with one pre-existing fluid channel 108 for all the surfaces may not provide adequate and/or efficient irrigation at the wound site. The number of pre-existing fluid channels 108 can be varied to achieve the desired irrigation coverage area.

FIG. 3 depicts a kit 310 including a flow control device 300 in a sealed environment, in accordance with some embodiments. The flow control device 300 is the same or similar to the flow control device 100.

The kit includes the flow control device 300 in a sealed package 320 with a top layer 322 and a bottom layer 324. The sealed package 320 provides a sterile environment 330 for the flow control device 300. As shown in FIG. 3, the flow control device 300 is in the sterile environment 330 of the sealed package 320. The sealed package 320 can maintain the sterility of the flow control device 300 until the flow control device 300 is needed. For example, the kit 310 can be kept in a medical environment until needed.

In some embodiments, the top layer 322 and the bottom layer 324 of the sealed package 320 can be plastic or other material. The top layer 322 and the bottom layer 324 can be sealed together to provide the sterile environment 330. When a need arises for the flow control device 300, the sealed package 320 can be opened. In some embodiments, the top layer 322 and the bottom layer 324 can be peeled apart to open the sealed package 320. Other methods can be used to open the sealed package 320 (e.g., use a scissors to open the sealed package 320).

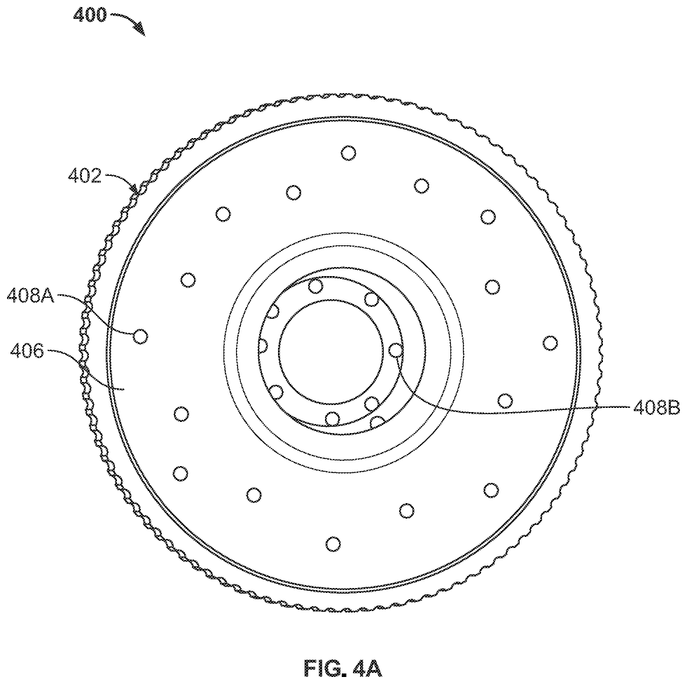

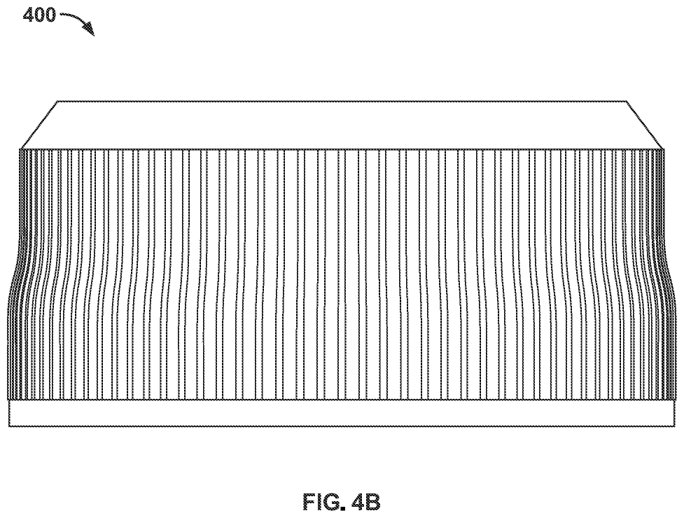

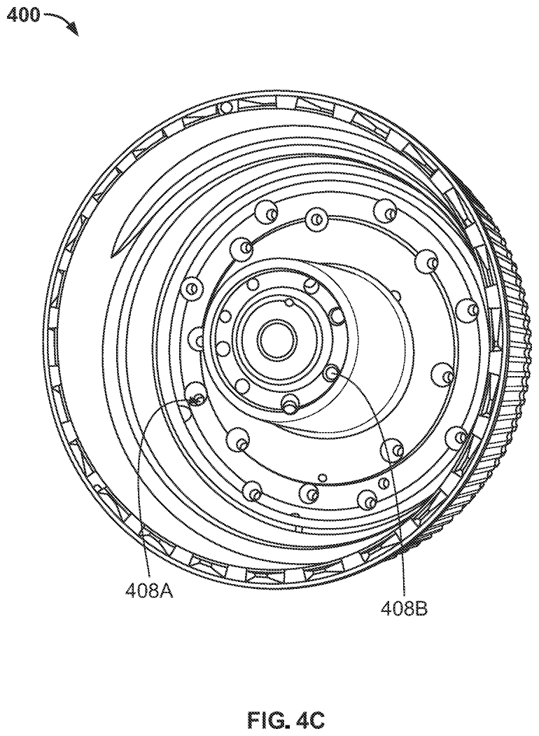

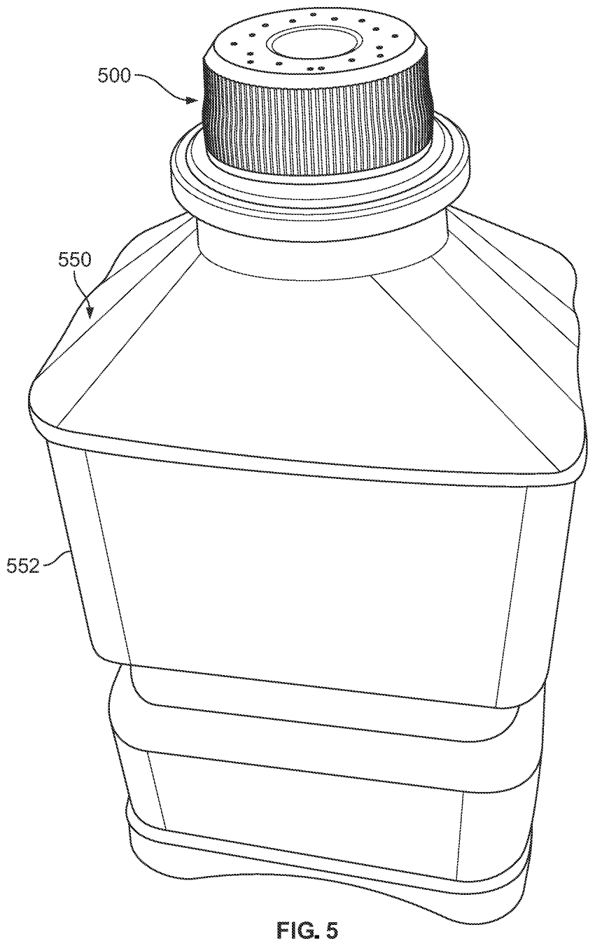

FIG. 4A is a top view of a flow control device 400, in accordance with some embodiments. FIG. 4B is a side view of the flow control device 400 of the present disclosure in FIG. 4A, in accordance with some embodiments. FIG. 4C is a bottom view of the flow control device 400 of the present disclosure in FIG. 4A, in accordance with some embodiments. The flow control device 400 is similar to the flow control device 100. Accordingly, similar reference numbers are used. Some of the differences between the flow control devices 400 and 100 are discussed. However, it will be appreciated that other differences may exist without departing from the scope of this disclosure. One example difference, as shown in FIG. 4B, is that there are no pre-existing fluid channels on the side of the flow control device 400. The flow control device 400 includes a body 402 with a structure 406. In some embodiments, the structure 406 defines a first plurality of pre-existing fluid channels 408A and a second plurality of pre-existing fluid channels 408B.

FIG. 5 is a top perspective view of a flow control device 500 attached to a container 550, in accordance with some embodiments. The flow control device 500 is similar to the flow control devices 100 and 200. The container 550 is similar to the container 250. Accordingly, similar reference numbers are used. As discussed herein, the container 550 can be any apparatus capable of holding a fluid. In some embodiments, the container 550 is defined by a main body 552 of the container 550. The flow control device 500 connects to the main body 552.

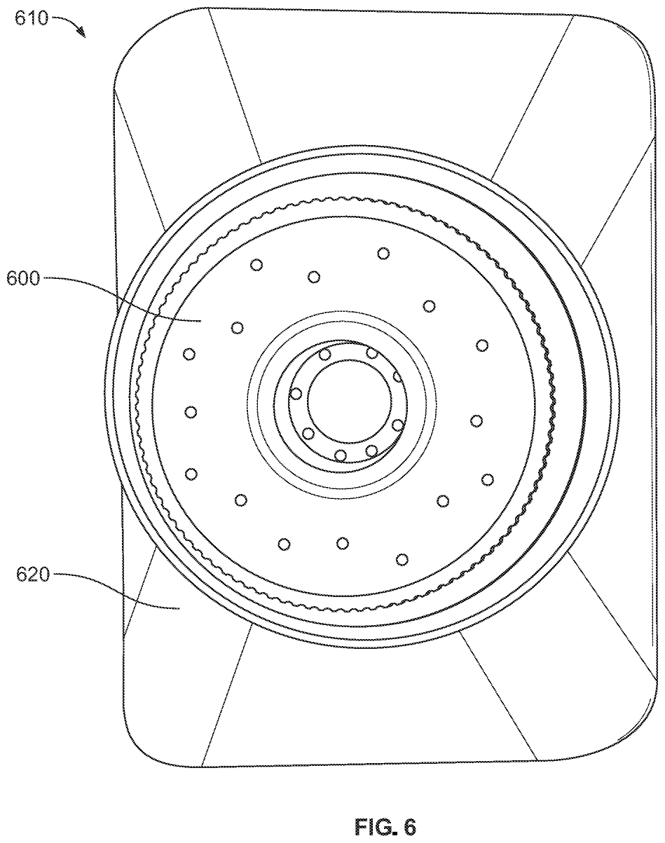

FIG. 6 depicts a kit 610 including a flow control device 600 in a sealed environment, in accordance with some embodiments. The kit 610 is similar to the kit 310. Accordingly, similar reference numbers are used. A sealed package 620 creates a sealed environment for the kit 610. The flow control device 600 resides within the sealed environment of the sealed package 620.

In some embodiments, the cap (such as any of the flow control devices described herein, e.g., flow control device 100) on the bottle (such as any of the containers described herein, e.g., container 550) does not have pre-existing fluid channels. Instead, pre-existing fluid channels can be created by a healthcare professional (e.g., using a sharp object to create a fluid channel in the cap).

In embodiments with pre-existing fluid channels (such as any of the pre-existing fluid channels described herein, e.g., at least one or more pre-existing fluid channels 108), securing the cap (such as any of the flow control devices described herein, e.g., flow control device 100) to a bottle (such as any of the containers described herein, e.g., container 550) removes the need for healthcare providers to use a sharp object (e.g., a needle) to create fluid channels in a saline/water container, thereby reducing potential injury to the healthcare provider. Additionally, by providing a cap (e.g., a sterilized cap such as sterilized flow control device 100) with pre-existing fluid channels that can securely attach onto the saline/water container, the healthcare provider does not lose sterility by inserting an object (e.g., a syringe) into the container to deliver boluses of fluid to the wound site. Re-inserting an object into the container presents an opportunity to lose the sterility of the fluid of the container and introduces the risk of cross-contamination. For the present disclosure, the cap, such as a sterilized cap, can be attached to the container and the contents of the container can be dispensed without the need to insert and reinsert an object into the container.

### ASPECTS

Various Aspects are described below. It is to be understood that any one or more of the features recited in the following Aspect(s) can be combined with any one or more other Aspect(s).

Aspect 1. A flow control device comprising: a body with a connecting portion, wherein the body comprises: a structure defining at least one or more pre-existing fluid channels, wherein the at least one or more pre-existing fluid channels are configured to control a fluid flow rate of a fluid exiting the body, and wherein controlling the fluid flow rate comprises controlling a predetermined pressure of the fluid flow rate; and wherein the connecting portion is configured to attach to a container.

Aspect 2. The flow control device of Aspect 1, wherein the predetermined pressure ranges from about 5 psi to about 15 psi.

Aspect 3. The flow control device of Aspect 1 or 2, wherein the body comprises a first surface and a second surface.

Aspect 4. The flow control device of Aspect 3, wherein the at least one or more pre-existing fluid channels comprise a first plurality of pre-existing fluid channels on the first surface and a second plurality of pre-existing fluid channels on the second surface.

Aspect 5. The flow control device of Aspect 4, wherein a cross-sectional area of each fluid channel of the first plurality of pre-existing fluid channels is the same.

Aspect 6. The flow control device of Aspect 4 or 5, wherein a cross-sectional area of each fluid channel of the first plurality of pre-existing fluid channels is different than a cross-sectional area of each fluid channel of the second plurality of pre-existing fluid channels.

Aspect 7. The flow control device as in any of the Aspects, wherein the second surface is at least partially recessed on the body defining an interior well on the body.

Aspect 8. The flow control device as in any of the Aspects, wherein the second surface of the body is substantially perpendicular to a longitudinal axis of the body.

Aspect 9. The flow control device as in any of the Aspects, wherein the flow control device is configured to be used in a medical, sterile environment.

Aspect 10. The flow control device as in any of the Aspects, wherein the connecting portion is threaded.

Aspect 11. A container comprising the flow control device as in any of the Aspects.

Aspect 12. The container of Aspect 11, wherein the container is configured to contain the fluid prior to the fluid exiting the body.

Aspect 13. A container comprising: a main body; a flow control device connected to the main body, wherein the flow control device comprises: a body of the flow control device with a connecting portion, wherein the body of the flow control device comprises: a structure defining at least one or more pre-existing fluid channels, wherein the at least one or more pre-existing fluid channels are configured to control a fluid flow rate of a fluid exiting the body of the flow control device, and wherein controlling the fluid flow rate comprises controlling a predetermined pressure of the fluid flow rate; and wherein the connecting portion is configured to attach to the container.

Aspect 14. The container of Aspect 13, wherein the container is configured to contain the fluid.

Aspect 15. A method comprising: connecting a fluid control device to a container, wherein the fluid control device comprises: a body with a connecting portion, wherein the body comprises: a structure defining at least one or more pre-existing fluid channels, wherein the at least one or more pre-existing fluid channels are configured to control a fluid flow rate of a fluid exiting the body, and wherein controlling the fluid flow rate comprises controlling a predetermined pressure of the fluid flow rate; and wherein the connecting portion is configured to attach to the container; and delivering the fluid to a site from the container at a controlled fluid flow rate.

Aspect 16. The method of Aspect 15, wherein delivering the fluid to the site from the container at the controlled fluid flow rate comprises deforming the container.

Aspect 17. The method of Aspect 16, wherein deforming the container comprises compressing the container.

Aspect 18. The method as in any of the Aspects, wherein delivering the fluid to the site from the container at the controlled fluid flow rate comprises delivering the fluid at the predetermined pressure.

Aspect 19. The method of Aspect 18, wherein the predetermined pressure ranges from about 5 psi to about 15 psi.

Aspect 20. A method comprising: connecting a fluid control device to a container holding a sterile fluid, wherein the fluid control device comprises: a body with a connecting portion, wherein the body comprises: a structure defining at least one or more pre-existing fluid channels, wherein the connecting portion is configured to attach to the container; delivering the sterile fluid to a site from the container at a controlled fluid flow rate while maintaining sterility of the sterile fluid.

Aspect 21. The method of Aspect 20, wherein delivering the sterile fluid to the site comprises delivering the sterile fluid to an irrigation coverage area of the site ranging from 5,000 mm² to about 45,000 mm².

Aspect 22. A kit comprising: a flow control device comprising: a body with a connecting portion, wherein the body comprises: a structure defining at least one or more pre-existing fluid channels, wherein the at least one or more pre-existing fluid channels are configured to control a fluid flow rate of a fluid exiting the body, and wherein controlling the fluid flow rate comprises controlling a predetermined pressure of the fluid flow rate; and wherein the connecting portion is configured to attach to a container; and a sealed package providing a sterile environment, wherein the flow control device is in the sterile environment of the sealed package.

Aspect 23. The kit of Aspect 22, wherein the body comprises a first surface and a second surface.

Aspect 24. The kit of Aspect 23, wherein the at least one or more pre-existing fluid channels comprise a first plurality of pre-existing fluid channels on the first surface and a second plurality of pre-existing fluid channels on the second surface.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

What is claimed is:

1. A flow control device comprising:
a body with a connecting portion, wherein the body comprises:
a structure defining at least one or more pre-existing fluid channels, wherein the at least one or more pre-existing fluid channels are configured to control a fluid flow rate of a fluid exiting the body, and wherein controlling the fluid flow rate comprises controlling a predetermined pressure of the fluid flow rate; and
wherein the connecting portion is configured to attach to a container.

2. The flow control device of claim 1,
wherein the predetermined pressure ranges from about 5 psi to about 15 psi.

3. The flow control device of claim 1,
wherein the body comprises a first surface and a second surface.

4. The flow control device of claim 3,
wherein the at least one or more pre-existing fluid channels comprise a first plurality of pre-existing fluid channels on the first surface and a second plurality of pre-existing fluid channels on the second surface.

5. The flow control device of claim 4,
wherein a cross-sectional area of each fluid channel of the first plurality of pre-existing fluid channels is the same.

6. The flow control device of claim 4,
wherein a cross-sectional area of each fluid channel of the first plurality of pre-existing fluid channels is different than a cross-sectional area of each fluid channel of the second plurality of pre-existing fluid channels.

7. The flow control device of claim 3,
wherein the second surface is at least partially recessed on the body defining an interior well on the body.

8. The flow control device of claim 3,
wherein the second surface of the body is substantially perpendicular to a longitudinal axis of the body.

9. The flow control device of claim 1,
wherein the flow control device is configured to be used in a medical, sterile environment.

10. The flow control device of claim 1,
wherein the connecting portion is threaded.

11. A container comprising the flow control device of claim 1.

12. The container of claim 11,
wherein the container is configured to contain the fluid prior to the fluid exiting the body.

13. A method comprising:
connecting a fluid control device to a container, wherein the fluid control device comprises:
a body with a connecting portion, wherein the body comprises:
a structure defining at least one or more pre-existing fluid channels, wherein the at least one or more pre-existing fluid channels are configured to control a fluid flow rate of a fluid exiting the body, and
wherein controlling the fluid flow rate comprises controlling a predetermined pressure of the fluid flow rate; and
wherein the connecting portion is configured to attach to the container; and
delivering the fluid to a site from the container at a controlled fluid flow rate.

14. The method of claim 13,
wherein delivering the fluid to the site from the container at the controlled fluid flow rate comprises deforming the container.

15. The method of claim 14,
wherein deforming the container comprises compressing the container.

16. The method of claim 13,
wherein delivering the fluid to the site from the container at the controlled fluid flow rate comprises delivering the fluid at the predetermined pressure.

17. The method of claim 16,
wherein the predetermined pressure ranges from about 5 psi to about 15 psi.

18. A kit comprising:
a flow control device comprising:
a body with a connecting portion, wherein the body comprises:
a structure defining at least one or more pre-existing fluid channels, wherein the at least one or more pre-existing fluid channels are configured to control a fluid flow rate of a fluid exiting the body, and
wherein controlling the fluid flow rate comprises controlling a predetermined pressure of the fluid flow rate; and
wherein the connecting portion is configured to attach to a container; and
a sealed package providing a sterile environment, wherein the flow control device is in the sterile environment of the sealed package.

19. The kit of claim 18,
wherein the body comprises a first surface and a second surface.

20. The kit of claim 19,
wherein the at least one or more pre-existing fluid channels comprise a first plurality of pre-existing fluid channels on the first surface and a second plurality of pre-existing fluid channels on the second surface.
